(19) United States
(12) Patent Application Publication
(10) Pub. No.: US 2023/0330149 A1
(43) Pub. Date: Oct. 19, 2023

(54) ENHANCEMENT OF CARTILAGE REGENERATIVE ACTIVITY OF STEM CELL POPULATIONS BASED ON REDUCTION OF INTRA-ARTICULAR CELLULAR MATERIAL

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Phoenix, AZ (US)

(73) Assignee: CREATIVE MEDICAL TECHNOLOGIES, INC., Phoenix, AZ (US)

(21) Appl. No.: 18/300,210

(22) Filed: Apr. 13, 2023

Related U.S. Application Data
(60) Provisional application No. 63/331,179, filed on Apr. 14, 2022.

(51) Int. Cl. A61K 35/28; A61K 38/47; A61K 38/48

(52) U.S. Cl. CPC A61K 35/28; A61K 38/47; A61K 38/4886; C12Y 302/01035; A61K 2035/124

(57) ABSTRACT

Methods of enhancing efficacy of cartilage regenerative therapies through reducing particulate and/or cellular debris residing in the intra-articular space. In one embodiment the invention teaches administration of low-dose protease and/or matrix metalloprotease and/or hyaluronidase prior to administration of stem cells in order to clear tissue of debris capable of suppressing regenerative activity of stem cells. In one embodiment of the invention clinical grade hyaluronidase is administered intra-articular in a patient receiving bone marrow aspirate/mononuclear cell therapy.

### CROSS-REFERENCE TO RELATED APPLICATIONS

This application claims priority to U.S. Provisional Application Ser. No. 63/331,179, titled “Enhancement of Cartilage Regenerative Activity of Stem Cell Populations Based on Reduction of Intra-Articular Cellular Material”, filed Apr. 14, 2022, which is hereby incorporated by reference in its entirety.

### FIELD OF THE INVENTION

The teachings herein are directed to methods of enchancing stem cells in order to administer to patients suffering from cartilage degeneration.

### BACKGROUND

It is accepted that osteoarthritis is the most common chronic musculoskeletal disorder. This conditioned afflicts nearly 23 million patients or 9% of the U.S. population. Arthritis is the leading age-related medical condition among women and ranks as the second most common such condition among men over 45 years of age. Deformities or orthopaedic joint impairment rank sixth among chronic disorders causing activity limitations. As an age-related condition, the continued projected growth of the elderly as a percentage of the total population will increase the prevalence of arthritis. Increasing longevity of the elderly population will further accelerate the incidence of age-related conditions such as arthritis. A significant portion of elderly arthritis sufferers are afflicted seriously enough to be considered disabled. Through the year 2000, the disabled elderly population is expected to increase to over 7 million (18% of elderly) patients, and more than double to 15-20 million over the subsequent fifty years.

Osteoarthritis is usually a primary degenerative process, result from childhood hip disorders, or as secondary to adult injury, infection, endocrine/metabolic disorders or bone dysplasia. Depending on the patient's age, their range of hip motion and clinical presentation, current operative procedures range from arthrodesis in young patients and osteotomy in patients under 60 years with reasonable hip motion, to hemi-, total, resection and cup arthroplasty. Knee osteoarthritis is characterized by pain, joint swelling, stiffness, motion loss and eventually deformity. As with the hip, knee osteoarthritis may be a primary degenerative process or result from a single or repeated knee injuries. The fact is recognized that osteoarthritis is a progressively degenerative disease, resulting in increasing pain, impairment and ultimately disability. While the available treatments seek to ameliorate pain or improve mobility, these treatments rarely modify the course of the disease, but rather attend to its consequences. For early stage osteoarthritis, treatment is largely limited to addressing the symptoms of inflammation with non-steroidal anti-inflammatory drugs, steroids for acute exacerbation and some use of the more toxic Disease-Modifying Arthorheumatic Drugs (DMARDS, e.g. gold salts, penicillamine, and methotrexate). Clinical reports indicate that even the newest DMARDS, such as tenidap, will not materially improve the clinical outcomes. None of these treatments stop the progression of the condition nor regenerate damaged cartilage.

### SUMMARY

Preferred embodiments are directed to methods of inhibiting, ameliorating or reversing cartilage degeneration comprising the steps of: a) identifying a patient in need of therapy; b) providing an intra-articular administration of an agent capable of decreasing debris found in the articular space; and c) administering a cellular population possessing regenerative properties.

Preferred methods include embodiments wherein said patient in need of therapy suffers from osteoarthritis.

Preferred methods include embodiments wherein identification of a patient in need of therapy is performed by quantification of range of motion of the afflicted joint.

Preferred methods include embodiments wherein identification of a patient in need of therapy is performed by imaging of the afflicted joint.

Preferred methods include embodiments wherein identification of a patient in need of therapy is performed by assessing cartilage erosion.

Preferred methods include embodiments wherein said assessment of said cartilage erosion is performed using magnetic resonance imaging.

Preferred methods include embodiments wherein said assessment of said cartilage erosion is performed using positron emission tomography.

Preferred methods include embodiments wherein said assessment of said cartilage erosion is performed using positron emission tomography.

Preferred methods include embodiments wherein said agent capable of decreasing debris found in the articular space is hyaluronidase.

Preferred methods include embodiments wherein said hyaluronidase is recombinant.

Preferred methods include embodiments wherein said hyaluronidase is derived from natural sources.

Preferred methods include embodiments wherein said agent capable of decreasing debris found in the articular space is a protease.

Preferred methods include embodiments wherein said protease is a matrix metalloprotease.

Preferred methods include embodiments wherein said protease is a gelatinase.

Preferred methods include embodiments wherein said protease is a collagenase.

Preferred methods include embodiments wherein said protease is a hydroxylase.

Preferred methods include embodiments wherein said matrix metalloprotease is MMP1.

Preferred methods include embodiments wherein said matrix metalloprotease is MMP2.

Preferred methods include embodiments wherein said matrix metalloprotease is MMP3.

Preferred methods include embodiments wherein said matrix metalloprotease is MMP7.

Preferred methods include embodiments wherein said matrix metalloprotease is MMP9.

Preferred methods include embodiments wherein said matrix metalloprotease is MMP13.

Preferred methods include embodiments wherein said cell population is autologous.

Preferred methods include embodiments wherein said cell population is allogeneic.

Preferred methods include embodiments wherein said cell population is xenogenic.

Preferred methods include embodiments wherein said cell population is bone marrow aspirate/mononuclear cells.

Preferred methods include embodiments wherein said cell population is mesenchymal stem cells.

Preferred methods include embodiments wherein said cell population is amniotic stem cells.

Preferred methods include embodiments wherein said cell population is embryonic stem cells.

Preferred methods include embodiments wherein said cell population is inducible pluripotent stem cells.

Preferred methods include embodiments wherein said cell population is a hematopoietic stem cell population.

Preferred methods include embodiments wherein said mesenchymal stem cells express CD90.

Preferred methods include embodiments wherein said mesenchymal stem cells express CD105.

Preferred methods include embodiments wherein said mesenchymal stem cells express c-met.

Preferred methods include embodiments wherein said mesenchymal stem cells express CD133.

Preferred methods include embodiments wherein said mesenchymal stem cells express c-kit.

Preferred methods include embodiments wherein said mesenchymal stem cells express IL-1 receptor.

Preferred methods include embodiments wherein said mesenchymal stem cells express IL-1 receptor antagonist when treated with interferon gamma.

Preferred methods include embodiments wherein said interferon gamma is administered at a concentration of 5 ng/ml for a period of 1 hour to 24 hours.

Preferred methods include embodiments wherein said agents are administered together with a growth factor.

Preferred methods include embodiments wherein said growth factor is hepatocyte growth factor.

Preferred methods include embodiments wherein said growth factor is epidermal growth factor.

Preferred methods include embodiments wherein said growth factor is insulin growth factor.

Preferred methods include embodiments wherein said growth factor is keratinocyte growth factor.

Preferred methods include embodiments wherein said cell population is administered before or after intraarticular laser treatment.

### DETAILED DESCRIPTION OF THE INVENTION

The invention focuses on the enhancement of therapeutic activity of stem cells, in one embodiment bone marrow aspirate and/or stem cells, on treatment of cartilage degenerative disorders. It is accepted that mesenchymal stem cell (MSC) technology provides opportunity to regenerate cartilage. The regeneration of cartilage and other injured or diseased tissue is achieved by administration of an optimal number of human mesenchymal stem cells to the repair site in an appropriate biomatrix delivery device, without the need for a second surgical site to harvest normal tissue grafts. The inventio provides means of enhancing therapeutic activity by the utilization of “regenerative adjuvants” which are capable of “cleaning up” the cartilage microenvironment before stem cells are administered in a patient in need of treatment. In the preferred embodiment, cartilage damaged as part of the degenerative effects of osteoarthritis, the present inventors have found that the human mesenchymal stem cell, such as those found in bone marrow, when combined with agents such as hyaluronidase, makes it possible to: (1) regenerate both shallow cartilage chondral defects and full thickness cartilage defects (osteochondral lesions); (2) broaden the suitable clinical population to routinely include middle-aged patients; (3) eliminate the use of autologous tissue grafts (mature cartilage and the periosteal covering) to repair an articular cartilage injury; (4) regenerate other types of injured cartilage such as patellar and spinal disk cartilage; (5) regenerate articular joint cartilage in older patients with osteoarthritis; and (6) form new cartilage and subchondral bone which fully integrate into the adjacent normal tissue. So, for example, in one embodiment, developing the present invention focused on the use of autologous mesenchymal stem cells for the regeneration of stable hyaline cartilage in affected joints. The articular cartilage of the knee and hip joints was the target of initial focus because the greatest morbidity and debilitating conditions in osteoarthritis arise from degeneration or degradation of these joints in the leg. The most promising approach to articular cartilage repair appears to be the use of autologous mesenchymal stem cells, which are osteochondral precursors. Mesenchymal stem cells for articular cartilage repair are combined with a controlled-resorption biodegradable matrix, preferably collagen-based products. These mesenchymal stem cell-matrix implants initiate, de novo, tissue formation, and maintain and stabilize the articular defect during the repair process. In addition to gels, the types of biomatrix materials that may be used include sponges, foams or porous fabrics that form a three-dimensional scaffold for the support of mesenchymal stem cells. These materials may be composed of collagen, gelatin, hyaluronan or derivatives thereof, or may consist of synthetic polymers, or may consist of composites of several different materials. The different matrix configurations and collagen formulations will depend on the nature of the cartilage defect, and include those for both open surgical and arthroscopic procedures. The invention provides several formulations of autologous, and/or allogeneic stem cells that serve as the basis of therapies for osteoarthritis are contemplated, depending on the stage, joint location, and severity of the disease. They are (1) a gel formulation that can be applied to osteochondral defects during arthroscopy; (2) an injectable cell suspension for delivery directly to the synovial space; and (3) a molded mesenchymal stem cell-biomatrix product to re-surface joint surfaces in advanced cases. The methods, compositions and implant devices of the invention are particularly suited for established conditions where superficial chondral or osteochondral defects can be diagnosed, but prior to the point where there is widespread joint instability and bone destruction. A characteristic indicator of chondral defect is a visibly altered gait or use of the joint to accommodate the discomfort or stiffness resulting from tissue damage, and the objective of treatment is to regenerate full thickness articular cartilage at the site of the defects to thereby prevent the joint destabilization and rapid joint destruction which are common sequelae of advanced osteoarthritis.

Patients ranging in age from 30-50 years with one or more well-defined articular cartilage lesions (as determined by imaging modalities or diagnostic arthroscopy) are ideal candidates for treatment in accordance with the invention. The need for advanced surgical intervention involving osteotomy or total joint arthroplasty can be deferred or even obviated. Administration is by application of autologous non-expanded, or culture-expanded (preferably autologous) human mesenchymal stem cells in a biodegradable collagen and/or fibrin matrix implant and/or blood serum clots to the affected joint. Application typically involves an arthroscopic procedure, which may include debridement of the defect prior to implantation of the human mesenchymal stem cell matrix. Within six to twelve weeks following implantation, the graft develops into fill thickness cartilage with complete bonding to the subchondral bone. Approximately a month prior to the initial treatment of the patient, a bone marrow aspirate (e.g., approximately 10-20 ml) is obtained from the patient's medial posterior iliac crest using standard aseptic techniques in an out-patient procedure. A Bone Marrow Collection and Transport Kit, described herein, provides most or all of the material needed for safe and efficient collection, identification, and transportation of the collected bone marrow. The double-sealed collection vessel is refrigerated until ready for human mesenchymal stem cell processing. A single aspirate sample can be culture-expanded sufficiently to provide material for multiple lesions (4-6) during one or several arthroscopic procedures. The cryopreservation techniques described herein permit retention of that portion of the aspirate that is not needed currently until it is required.

According to certain embodiments of the invention, we describe one or more doses of the preselected stem cells at preselected time intervals for administration concurrently with, or subsequent to administration of agents capable of removing articular debris. By way of nonlimiting example the preselected stem cells are delivered at approximately weekly intervals, at approximately two week intervals, at approximately three week intervals, at approximately monthly intervals, at approximately two month intervals, at approximately three month intervals, at approximately four month intervals, at approximately five month intervals, or at approximately six month intervals. In at least one embodiment, the therapeutic dose of each IV injection of preselected stem cells comprises about half a million stem cells per kg of the patient's body weight up to a maximum of fifty million stem cells regardless of body weight. In order to obtain the necessary number of stem cells, preselected stem cells are collected and expanded utilizing cell culture techniques described in further detail below, and those expanded stem cells are harvested and segregated into cell counts of approximately six million to about fifty million cells, as needed. As used herein, a therapeutic dose means the number of stem cells of sufficient quantity to decrease the physiological symptoms of the specified disease or diseased state in the patient. Thereafter, according to one embodiment, those segregated cells are diluted into a balanced saline solution or other suitable dilutive solution such that each diluted population of cells has a concentration of approximately 200,000 cells or less per mL of solution. According to yet another embodiment, each cell count is diluted to a concentration of approximately 1,000,000 cells or less per mL of solution; approximately 500,000 cells or less per mL of solution; approximately 250,000 cells or less per mL of solution; and approximately 100,000 cells or less per mL of solution. It will be appreciated that the diluted population of cells may be harvested such that they are free of substantially all of the culture medium upon which the stem cells were expanded, or the cells may be harvested to include at least a portion of the cell conditioned medium upon which the cells were expanded. Likewise, the diluted population of cells may comprise additional physiological electrolyte additives. Alternatively, the medium may also be delivered free of cells. In certain embodiments, preselected stem cells are autologous or allogeneic to the patient, and those preselected stem cells may be isolated from a donor during health procedures that are unrelated to the purpose of harvesting stem cells. As such, stem cells may be harvested in a manner that does not adversely affect the donor or result in unnecessary medical treatments to the donor. Further, it will be appreciated that donors are preferably screened to ensure that the donor is in good general health, and may be screened for the presence of diseases, current status of vaccinations, or presence of antibiotics in the donor's system as necessary. Stem cells include dental derived stem cells such as stem cells harvested from dental pulp, periodontal ligaments, and other dental tissues; stem cells harvested from testicle tissue; stem cells harvested from bone marrow; stem cells harvested from placental tissue; stem cells harvested from uterine tissue (including endometrial regenerative cells), stem cells harvested from umbilical cord tissue, and stem cells harvested from full thickness skin biopsies.

Numerous types of stem cells may be utilized for the practice of the invention. In one embodiment, bone marrow aspirate may be taken whole with no manipulation and utilized. Also, bone marrow may be collected and placed within a “washing vessel”. Before the collection procedure a “washing tube” is prepared in the class 100 Biological Safety Cabinet in a Class 10,000 GMP Clean Room. To prepare the washing tube, 0.2 mL amphotericin B (Sigma-Aldrich, St Louis, Mo.), 0.2 mL penicillin/streptomycin (Sigma 50 ug/nl) and 0.1 mL EDTA-Na2 (Sigma) is added to a 50 mL conical tube (Nunc) containing 40 mL of GMP-grade phosphate buffered saline (PBS). Specifically, the washing tube containing the collected bone marrow is topped up to 50 mL with PBS in a class 100 Biological Safety Cabinet and cells are washed by centrifugation at 500 g for 10 minutes at room temperature, which yields a cell pellet at the bottom of the conical tube. Under sterile conditions supernatant is decanted and the cell pellet is gently dissociated by tapping until the pellet appeared liquid. The pellet is re-suspended in 25 mL of PBS and gently mixed so as to produce a uniform mixture of cells in PBS. In order to purify mononuclear cells, 15 mL of Ficoll-Paque (Fisher Scientific, Portsmouth N.H.) density gradient is added underneath the cell-PBS mixture using a 15 mL pipette. The mixture is subsequently centrifuged for 20 minutes at 900 g. Thereafter, the buffy coat is collected and placed into another 50 mL conical tube together with 40 mL of PBS. Cells are then centrifuged at 400 g for 10 minutes, after which the supernatant is decanted and the cell pellet re-suspended in 40 mL of PBS and centrifuged again for 10 minutes at 400 g. The cell pellet is subsequently re-suspended in 5 mL complete DMEM-low glucose media (GibcoBRL, Grand Island, N.Y.) supplemented with 20% Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments. Additionally, the media is supplemented with 1% penicillin/streptomycin, 1% amphotericin B, and 1% glutamine. The re-suspended cells are mononuclear cells substantially free of erythrocytes and polymorphonuclear leukocytes as assessed by visual morphology microscopically. Viability of the cells is assessed with trypan blue. Only samples with >90% viability were selected for cryopreservation in sealed vials. In some embodiments of the invention cells are expanded outside of the body. This may be to increase the number of cells and/or activity of the cells. Cell expansion for cells can be performed in a clean room facilities purpose built for cell therapy manufacture and meeting GMP clean room classification. In a sterile class II biologic safety cabinet located in a class 10,000 clean production suite, cells are thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used was sequestered and one lot was used for all experiments.

Cells were subsequently placed in a T-225 flask containing 45 mL of cDMEM and cultured for 24 hours at 37.degree. C. at 5% CO2 in a fully humidified atmosphere. This allowed the MSC to adhere. Non-adherent cells were washed off using cDMEM by gentle rinsing of the flask. Adherent cells are subsequently detached by washing the cells with PBS and addition of 0.05% trypsin containing EDTA (Gibco, Grand Island, N.Y., USA) for 2 minutes at 37.degree. C. at 5% CO2 in a fully humidified atmosphere. Cells are centrifuged, washed and plated in T-225 flask in 45 mL of cDMEM. In one embodiment this procedure results in generation of approximately 6 million cells per initiating T-225 flask. The cells of the first flask were then split into 4 flasks. Cells are grown for approximately 4 days after which approximately 6 million cells per flask were present (24 million cells total). All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. In some embodiments, donor cells were collected in sterile conditions, shipped to a contract manufacturing facility, assessed for lack of contamination and expanded. The expanded cells were stored in cryovials of approximately 6 million cells/vial, with approximately 100 vials per donor. At each step of the expansion quality control procedures were in place to ensure lack of contamination or abnormal cell growth. In another aspect, cells are grown in media and the cells, along with the media, are recovered after about 5-10 days. The cells are prepared in this “conditioned” media for transfusion at concentrations of less than about 100,000 cells per mL Physiological electrolyte additives may be added. The cell solution is administered intravenously. In another embodiment, cells are grown in media for about 5-10 days. This media is then transfused intravenously without cells or given locally to the site of the injury. Further methods involve isolation and/or concentration of stem cell produced factors and/or further refinements of these chemicals and/or compounds.

Administration of stem cell adjuvants, or regenerative adjuvants may be performed intravenously, or more preferably intra-articularly. The “hyaluronidase” being used for the present invention can be derived from any source whatsoever. For example, the hyaluronidase may be derived from a mammal such as from human, mouse, rat, pig, sheep or cow. For instance, the hyaluronidase may be recovered from bovine protein (bovine type), alternatively from leeches or bacteria (e.g. in the form of hyaluronate lyase). The hyaluronidase can also be of vegetable origin. The hyaluronidase can be isolated, for instance, from potatoes, tobaccos and peas. Purification, chemical synthesis and genetic engineering techniques including production in a transgenic host generally known in the art can likewise be used to produce hyaluronidase. Particularly preferred is any hyaluronidase which splits and thus depolymerises hyaluronic acid, chondroitin-4-sulphate, chondroitin-6-sulphate and mucotin sulphate where the most preferred hyaluronidase is an enzyme available commercially such as, by way of example, the hyaluronidase marketed under the trade name of “Hylase Dessau” by RIEMSER Arzneimittel AG, preferably Hylase Dessau 1500 IU by RIEMSER Arzneimittel AG, containing 1500 IU of bovine hyaluronidase. For prevention and/or treatment of the disorders of the present invention, a mixture of hyaluronidases of different origins can also be used. When using other hyaluronidases than Hylase Dessau other dosages may be required which the person skilled in the art can easily determine according to the practical circumstances of the case. According to a preferred embodiment, the hyaluronidase is used as a prevention or treatment of cartilage degeneration in a human or animal patient. According to yet another preferred embodiment, hyaluronidase is prepared for administration through the digestive tract, or parenterally. When administered through the digestive tract, hyaluronidase is preferably administered nasally, sublingually, orally, such as by way of a tablet, solution or capsule. Alternatively, hyaluronidase is to be administered parenterally, preferably through injection by intravenous, intraperitoneal, intracerebral (intraparenchymal), intracerebroventricular, intramuscular, subcutaneous, intraocular, intraarterial, intraportal, or intralesional routes; by sustained release systems or by implantation devices. Where desired, the compositions may be administered by bolus injection or continuously by infusion, or by implantation device. When parenteral administration is contemplated, the therapeutic compositions for use in this invention may be in the form of a pyrogen-free, parenterally acceptable, aqueous solution comprising the desired hyaluronidase in a pharmaceutically acceptable vehicle. A particularly suitable vehicle for parenteral injection is sterile distilled water in which a hyaluronidase is formulated as a sterile, isotonic solution, properly preserved, preferably as isotonic solution (saline, 0.9% NaCl solution). Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Other suitable means for the introduction of hyaluronidase include implantable drug delivery devices.

According to another aspect of the present invention, it is provided a pharmaceutical composition which comprises hyaluronidase, a composition of the present invention comprising hyaluronidase and at least one further matrix metalloprotease such as MMP-1, MMP-3, and MM)-9. Preferably, the pharmaceutical composition comprises a therapeutically effective amount of hyaluronidase and preferably also a therapeutically effective amount of the at least one further antihypertensive, in admixture with at least one pharmaceutically and/or physiologically acceptable formulation agent, at least one vehicle and/or at least one carrier, the formulation agent, the vehicle and the carrier selected for suitability with the mode of administration. Such acceptable formulation agents are generally known in the art and inter alia comprise agents for modifying, maintaining, or preserving, for example, the pH, osmolarity, viscosity, clarity, color, isotonicity, odor, sterility, stability, rate of dissolution or release, adsorption, or penetration of the composition. Suitable formulation agents include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides-preferably sodium or potassium chloride- or mannitol sorbitol), delivery vehicles, diluents, excipients and/or pharmaceutical adjuvants. See Remington's Pharmaceutical Sciences (18th Ed., A. R. Gennaro, ed., Mack Publishing Company 1990). The formulation components are present in concentrations that are acceptable to the site of administration. For example, buffers are used to maintain the composition at physiological pH or at a slightly lower pH, typically within a pH range of from about 5 to about 8. The optimal pharmaceutical composition according to the invention will be determined by a skilled artisan depending upon, for example, the intended route of administration, delivery format, and desired dosage. See, e. g., Remington's Pharmaceutical Sciences, supra. Such compositions may influence the physical state, stability, rate of in vivo release, and rate of in vivo clearance of the hyaluronidase.

The primary vehicle or carrier in a pharmaceutical composition according to the invention may be either aqueous or non-aqueous in nature. For example, a suitable vehicle or carrier for injection may be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute. In one embodiment of the present invention, hyaluronidase compositions may be prepared for storage by mixing the selected composition having the desired degree of purity with optional formulation agents (Remington's Pharmaceutical Sciences, supra) in the form of a lyophilized cake or an aqueous solution. Further, the pharmaceutical composition may be formulated as a lyophilizate using appropriate excipients such as sucrose.

According to a preferred embodiment, at least about 3,000 IU, about 4,500 IU, preferably at least about 6,000 IU, more preferably at least about 8,000 IU, even more preferably at least about 10,000 IU, most preferably at least about 15,000 IU, most preferably at least about 20,000 IU, most preferably at least about 25,000 IU, most preferably at least about 30,000 IU of hyaluronidase are administered per day.

Preferably, the Hyaluronidase dosage is increased over time until preferably a plateau dosage is reached, particularly in the initial period of the Hyaluronidase therapy. Preferably the Hyaluronidase dosage increases at an interval ranging from about 1 day to about 6 days, preferably from about 1 day to about 3 days; each dosage increase ranging from about 500 IU to about 2,500 IU, more preferably each dosage increase is about 1,500 IU, as exemplified in the dosage regimens of the Examples. Preferably, the Hyaluronidase dosage starts out from about 3,000 IU to about 4,500 IU, preferably from about 4,500 IU and is then increased over time to a dosage of about 10,000 IU to about 15,000 IU, preferable to a dosage of about 12,500 IU, preferably the dosage increases are carried out as described before. Of course the dosage increase may differ from day to day. According to a preferred embodiment the dosage may as well decrease over time, such as before the Hyaluronidase administration is discontinued.

Preferably the dose may be administered by a single administration or in more than one administration spread over 24 hours. Preferably, the expression “per day” is also meant to encompass the administration of the indicated dose on each and every day. More preferably, the term “per day” also encompasses the case where the indicated dose is to be administered on average per day, i.e. the dose may vary from day to day but the averaged dose is defined by the indicated dose “per day”. The expression “IU” as used herein preferably refers to the generally known term international unit (IU). According to a preferred embodiment hyaluronidase is to be administered from about 1 to about 7 days per week, preferably about 3 to about 7 days per week, more preferably about 5 to about 7 days per week, more preferably about 1 to about 5 days per week, more preferably about 3 to about 5 days per week. According to a preferred embodiment hyaluronidase is to be administered at least about 1 week, at least about 2 weeks, at least about 3 weeks, at least about 4 weeks, at least about 5 weeks, at least about 6 weeks, at least about 7 weeks, at least about 8 weeks, preferably at least about 1 month, at least about 2 months, at least about 3 months, at least about 4 months, at least about 5 months or at least about 6 months. Preferably the administration is carried out continuously over the indicated time period. Preferably, the at least about 3,000 IU hyaluronidase are to be administered per day for about 3 to about 7 days per week for at least about 2 weeks, preferably for at least about 4 weeks. Preferably the administration is carried out continuously over the indicated time period.

Of course, the concrete dosage duration can naturally be varied in each individual case and an effective treatment regimen has to be adapted to the type of cartilage degeneration which a particular patient suffers from. In addition, the effective medication of hyaluronidase for patients suffering from the same kind of cartilage degeneration may have to be adapted to the individual patient. According to yet another aspect of the invention it is provided a composition comprising hyaluronidase and at least one further cartilage degeneration treating drugs or approaches as defined below. It is generally known how to formulate the hyaluronidase and at least one further drug inhibiting cartilage degeneration. The dosage of the at least one further cartilage degeneration inhibiting drug can be readily determined by the skilled physician. Claims

1. A method of inhibiting, ameliorating or reversing cartilage degeneration comprising the steps of: a) identifying a patient in need of therapy; b) providing an intra-articular administration of an agent capable of decreasing debris found in the articular space; and c) administering a cellular population possessing regenerative properties.

2. The method of claim 1, wherein said patient in need of therapy suffers from osteoarthritis.

3. The method of claim 1, wherein identification of a patient in need of therapy is performed by assessing cartilage erosion.

4. The method of claim 3, wherein said assessment of said cartilage erosion is performed using magnetic resonance imaging.

5. The method of claim 1, wherein said agent capable of decreasing debris found in the articular space is hyaluronidase.

6. The method of claim 5, wherein said agent capable of decreasing debris found in the articular space is a protease.

7. The method of claim 6, wherein said protease is a matrix metalloprotease.

8. The method of claim 7, wherein said matrix metalloprotease is MMP13.

9. The method of claim 1, wherein said cell population is autologous.

10. The method of claim 1, wherein said cell population is allogeneic.

11. The method of claim 1, wherein said cell population is bone marrow aspirate/mononuclear cells.

12. The method of claim 1, wherein said cell population is mesenchymal stem cells.

13. The method of claim 1, wherein said cell population is inducible pluripotent stem cells.

14. The method of claim 12, wherein said mesenchymal stem cells express IL-1 receptor.

15. The method of claim 12, wherein said mesenchymal stem cells express IL-1 receptor antagonist when treated with interferon gamma.

16. The method of claim 15, wherein said interferon gamma is administered at a concentration of 5 ng/ml for a period of 1 hour to 24 hours.

17. The method of claim 1, wherein said agents are administered together with a growth factor.

18. The method of claim 17, wherein said growth factor is hepatocyte growth factor.

19. The method of claim 17, wherein said growth factor is epidermal growth factor.

20. The method of clam 1, wherein said cell population is administered before or after intraarticular laser treatment.
